(19) United States
(12) Patent Application Publication
(10) Pub. No.: US 2012/0309824 A1
(43) Pub. Date: Dec. 6, 2012

(54) AZEOTROPIC AND AZEOTROPE-LIKE COMPOSITIONS OF E-1,1,1,4,4,4-HEXAFLUORO-2-BUTENE AND ETHYLENE OXIDE AND USES THEREOF

(75) Inventors: Mark L. Robin, Middletown, DE (US); Ralph Newton Miller, Newark, DE (US)

(73) Assignee: E.I. DU PONT DE NEMOURS AND COMPANY, Wilmington, DE (US)

(21) Appl. No.: 13/574,802

(22) PCT Filed: Feb. 25, 2011
(86) PCT No.: PCT/US2011/026255

§ 371 Date: Jul. 24, 2012

Related U.S. Application Data
(60) Provisional application No. 61/307,953, filed on Feb. 25, 2010.

(51) Int. Cl. A01N 43/20; A01P 1/00; C08J 9/14; C09K 5/00; F25D 31/00; A62D 1/00; H01B 3/24; C09K 3/30; A61L 2/18; C11D 17/00; C09K 3/00

(52) U.S. Cl. 514/475; 510/411; 521/131; 252/67; 252/364; 252/78.1; 252/2; 252/580; 516/8; 516/12; 422/37; 62/119

(57) ABSTRACT

Azeotropic or azeotrope-like compositions are disclosed. The azeotropic or azeotrope-like compositions are mixtures of E-1,1,1,4,4,4-hexafluoro-2-butene and ethylene oxide. Also disclosed are double azeotropic compositions of E-1,1,1,4,4,4-hexafluoro-2-butene and ethylene oxide. Also disclosed is a process of preparing a thermoplastic or thermoset foam by using such azeotropic or azeotrope-like compositions as blowing agents. Also disclosed is a process of producing refrigeration by using such azeotropic or azeotrope-like compositions. Also disclosed is a process of using such azeotropic or azeotrope-like compositions as solvents. Also disclosed is a process of producing an aerosol product by using such azeotropic or azeotrope-like compositions. Also disclosed is a process of using such azeotropic or azeotrope-like compositions as heat transfer media. Also disclosed is a process of extinguishing or suppressing a fire by using such azeotropic or azeotrope-like compositions. Also disclosed is a process of using such azeotropic or azeotrope-like compositions as dielectrics. Also disclosed is a method for sterilizing an article by using such azeotropic or azeotrope-like compositions.

### BACKGROUND OF THE INVENTION

1. Field of the Disclosure

The present disclosure relates to azeotropic or azeotrope-like compositions of E-1,1,1,4,4,4-hexafluoro-2-butene and ethylene oxide. Additionally, the present disclosure relates to methods for sterilizing an article using azeotropic or azeotrope-like compositions consisting essentially of E-1,1,1,4,4,4-hexafluoro-2-butene and ethylene oxide.

2. Description of Related Art

Many industries have been working for the past few decades to find replacements for the ozone depleting chlorofluorocarbons (CFCs) and hydrochlorofluorocarbons (HCFCs). The CFCs and HCFCs have been employed in a wide range of applications, including their use as aerosol propellants, refrigerants, cleaning agents, sterilant mixtures, expansion agents for thermoplastic and thermoset foams, heat transfer media, gaseous dielectrics, fire extinguishing and suppression agents, power cycle working fluids, polymerization media, particulate removal fluids, carrier fluids, buffing abrasive agents, and displacement drying agents. In the search for replacements for these versatile compounds, many industries have turned to the use of hydrofluorocarbons (HFCs).

The HFCs do not contribute to the destruction of stratospheric ozone, but are of concern due to their contribution to the “greenhouse effect”, i.e., they contribute to global warming. As a result of their contribution to global warming, the HFCs have come under scrutiny, and their widespread use may also be limited in the future. Thus, there is a need for compositions that do not contribute to the destruction of stratospheric ozone and also have low global warming potentials (GWPs). Certain hydrofluoroolefins, such as 1,1,1,4,4,4-hexafluoro-2-butene (CF₃CH═CHCF₃, FO-1336mzz, FC-1336mzz), are believed to meet both goals.

### SUMMARY OF THE INVENTION

This disclosure provides azeotropic or azeotrope-like compositions consisting essentially of (a) E-FO-1336mzz and (b) ethylene oxide; wherein the ethylene oxide is present in an effective amount to form an azeotropic or azeotrope-like mixture with E-FO-1336mzz.

This disclosure also provides processes of using these azeotropic or azeotrope-like compositions as blowing agents, refrigerants, solvents, aerosol propellants, heat transfer medias, fire extinguishants, fire suppression agents, dielectrics, cleaning agents or sterilant mixtures.

### BRIEF SUMMARY OF THE DRAWINGS

FIG. 1-FIG. 1 is a graph of the vapor/liquid equilibrium curve (Px diagram) for mixtures of E-FO-1336mzz and ethylene oxide at about 20° C. It demonstrates the formation of double azeotropic and double azeotrope-like compositions consisting essentially of E-FO-1336mzz and ethylene oxide.

FIG. 2-FIG. 2 is a zoomed-in graph of FIG. 1 with mole fractions of E-FO-1336mzz between 0.2 and 0.8.

### DETAILED DESCRIPTION OF THE INVENTION

In many applications, the use of a pure single component or an azeotropic or azeotrope-like mixture is desirable. For example, when a blowing agent composition (also known as foam expansion agents or foam expansion compositions) is not a pure single component or an azeotropic or azeotrope-like mixture, the composition may change during its application in the foam forming process. Such change in composition could detrimentally affect processing or cause poor performance in the application. Also, in refrigeration applications, a refrigerant is often lost during operation through leaks in shaft seals, hose connections, soldered joints and broken lines. In addition, the refrigerant may be released to the atmosphere during maintenance procedures on refrigeration equipment. If the refrigerant is not a pure single component or an azeotropic or azeotrope-like composition, the refrigerant composition may change when leaked or discharged to the atmosphere from the refrigeration equipment. The change in refrigerant composition may cause the refrigerant to become flammable or to have poor refrigeration performance. Accordingly, there is a need for using azeotropic or azeotrope-like mixtures in these and other applications, for example azeotropic or azeotrope-like mixtures containing E-1,1,1,4,4,4-hexafluoro-2-butene (E-CF₃CH═CHCF₃, E-FO-1336mzz, E-FC-1336mzz).

Ethylene oxide is widely employed as a sterilizing agent in sterilization compositions; due to its flammability, ethylene oxide is generally not used alone for sterilization. Most often, ethylene oxide is employed in admixture with a fire suppression agent such as carbon dioxide or a fluorocarbon gas. CFCs were employed in ethylene oxide sterilant mixtures, but due to the ozone depleting properties of CFCs, the production and use of CFCs is being banned on a global basis. HFCs are also useful as components of sterilant mixtures with ethylene oxide. Some parties, however, have expressed concern over the moderate global warming potential of HFCs, and therefore it is desirable to identify new sterilant mixtures with reduced global warming potential, such as E-FO-1336mzz/ethylene oxide mixtures.

If the sterilant is not a pure single component or an azeotropic or azeotrope-like composition, the sterilant composition may change during the sterilizing processes. The change in sterilant composition may cause the sterilant to become flammable. Accordingly, there is a need for using azeotropic or azeotrope-like mixtures, for example azeotropic or azeotrope-like mixtures of ethylene oxide and E-FO-1336mzz.

Before addressing details of embodiments described below, some terms are defined or clarified.

FO-1336mzz may exist as one of two configurational isomers, E or Z. FO-1336mzz as used herein refers to the isomers, Z-FO-1336mzz or E-FO-1336mzz, as well as any combinations or mixtures of such isomers.

As used herein, the terms “comprises,” “comprising,” “includes,” “including,” “has,” “having” or any other variation thereof, are intended to cover a non-exclusive inclusion. For example, a process, method, article, or apparatus that comprises a list of elements is not necessarily limited to only those elements but may include other elements not expressly listed or inherent to such process, method, article, or apparatus. Further, unless expressly stated to the contrary, “or” refers to an inclusive or and not to an exclusive or. For example, a condition A or B is satisfied by any one of the following: A is true (or present) and B is false (or not present), A is false (or not present) and B is true (or present), and both A and B are true (or present).

Also, use of “a” or “an” are employed to describe elements and components described herein. This is done merely for convenience and to give a general sense of the scope of the invention. This description should be read to include one or at least one and the singular also includes the plural unless it is obvious that it is meant otherwise.

Unless otherwise defined, all technical and scientific terms used herein have the same meaning as commonly understood by one of ordinary skill in the art to which this invention belongs. Although methods and materials similar or equivalent to those described herein can be used in the practice or testing of embodiments of the present invention, suitable methods and materials are described below. All publications, patent applications, patents, and other references mentioned herein are incorporated by reference in their entirety, unless a particular passage is cited. In case of conflict, the present specification, including definitions, will control. In addition, the materials, methods, and examples are illustrative only and not intended to be limiting.

E-FO-1336mzz is a known compound, and its preparation method has been disclosed, for example, in Dawoodi, et. al., Journal of the Chemical Society, Chemical Communications (1982), (12). 696-8, hereby incorporated by reference in its entirety.

This application includes azeotropic or azeotrope-like compositions consisting essentially of (a) E-FO-1336mzz and (b) ethylene oxide; wherein the ethylene oxide is present in an effective amount to form an azeotropic or azeotrope-like mixture with E-FO-1336mzz.

By effective amount (to form an azeotropic or azeotrope-like combination) is meant an amount of ethylene oxide, which, when combined with E-FO-1336mzz, results in the formation of an azeotropic or azeotrope-like mixture. This definition includes the amounts of each component, which amounts may vary depending on the pressure applied to the composition so long as the azeotropic or azeotrope-like compositions continue to exist at the different pressures, but with possible different boiling points. Therefore, effective amount includes the amounts, such as may be expressed in weight or mole percentages, of each component of the compositions of the instant invention which form azeotropic or azeotrope-like compositions at temperatures or pressures other than as described herein.

As recognized in the art, an azeotropic composition is an admixture of two or more different components which, when in liquid form under a given pressure, will boil at a substantially constant temperature, which temperature may be higher or lower than the boiling temperatures of the individual components, and which will provide a vapor composition essentially identical to the overall liquid composition undergoing boiling. (see, e.g., M. F. Doherty and M. F. Malone, Conceptual Design of Distillation Systems, McGraw-Hill (New York), 2001, 185-186, 351-359).

Accordingly, the essential features of an azeotropic composition are that at a given pressure, the boiling point of the liquid composition is fixed and that the composition of the vapor above the boiling composition is essentially that of the overall boiling liquid composition (i.e., no fractionation of the components of the liquid composition takes place). It is also recognized in the art that both the boiling point and the weight percentages of each component of the azeotropic composition may change when the azeotropic composition is subjected to boiling at different pressures. Thus, an azeotropic composition may be defined in terms of the unique relationship that exists among the components or in terms of the compositional ranges of the components or in terms of exact weight percentages of each component of the composition characterized by a fixed boiling point at a specified pressure.

By “double azeotropic” composition is meant that at a certain temperature there are two different azeotropic compositions for an admixture of two or more different components. The P-x (pressure versus composition) diagram for a double azeotrope exhibits both a minimum and a maximum pressure. Similarly, the T-x (temperature versus composition) diagram for a double azeotrope exhibits both a minimum and a maximum temperature. Some double azeotropes have been described in “Double azeotropy in binary mixtures of NH₃ and CHF₂CF₃”, Fluid Phase Equilibria, volume 127, on pages 191 to 203 (1997) by Kao, et. al., and in “Double Azeotropy in the Benzene+Hexafluorobenzene System”, J. Chem. Eng. Data, volume 41, on pages 21 to 24 (1996) by A. Aucejo, et. al.. Both aforementioned references are hereby incorporated by reference.

For the purpose of this invention, an azeotrope-like composition means a composition that behaves like an azeotropic composition (i.e., has constant boiling characteristics or a tendency not to fractionate upon boiling or evaporation). Hence, during boiling or evaporation, the vapor and liquid compositions, if they change at all, change only to a minimal or negligible extent. This is to be contrasted with non-azeotrope-like compositions in which during boiling or evaporation, the vapor and liquid compositions change to a substantial degree.

Additionally, azeotrope-like compositions exhibit dew point pressure and bubble point pressure with virtually no pressure differential. That is to say that the difference in the dew point pressure and bubble point pressure at a given temperature will be a small value. In this invention, compositions with a difference in dew point pressure and bubble point pressure of less than or equal to 5 percent (based upon the bubble point pressure) is considered to be azeotrope-like.

It is recognized in this field that when the relative volatility of a system approaches 1.0, the system is defined as forming an azeotropic or azeotrope-like composition. Relative volatility is the ratio of the volatility of component 1 to the volatility of component 2. The ratio of the mole fraction of a component in vapor to that in liquid is the volatility of the component.

To determine the relative volatility of any two compounds, a method known as the PTx method can be used. In this procedure, the total absolute pressure in a cell of known volume is measured at a constant temperature for various compositions of the two compounds. Use of the PTx Method is described in detail in “Phase Equilibrium in Process Design”, Wiley-Interscience Publisher, 1970, written by Harold R. Null, on pages 124 to 126; hereby incorporated by reference.

These measurements can be converted into equilibrium vapor and liquid compositions in the PTx cell by using an activity coefficient equation model, such as the Non-Random, Two-Liquid (NRTL) equation, to represent liquid phase nonidealities. Use of an activity coefficient equation, such as the NRTL equation is described in detail in “The Properties of Gases and Liquids,” 4th edition, published by McGraw Hill, written by Reid, Prausnitz and Poling, on pages 241 to 387, and in “Phase Equilibria in Chemical Engineering,” published by Butterworth Publishers, 1985, written by Stanley M. Walas, pages 165 to 244. Both aforementioned references are hereby incorporated by reference. Without wishing to be bound by any theory or explanation, it is believed that the NRTL equation, together with the PTx cell data, can sufficiently predict the relative volatilities of the E-1,1,1,4,4,4-hexafluoro-2-butene/ethylene oxide mixtures of the present invention and can therefore predict the behavior of these mixtures in multi-stage separation equipment such as distillation columns.

It was found through experiments that E-FO-1336mzz and ethylene oxide form double azeotropic or double azeotrope-like compositions at about 20° C. and about 70° C.

To determine the relative volatility of this binary pair, the PTx method described above was used. The total absolute pressure in a Fix cell of known volume was measured at constant temperature for various binary compositions. These measurements were then reduced to equilibrium vapor and liquid compositions in the cell using the NRTL equation.

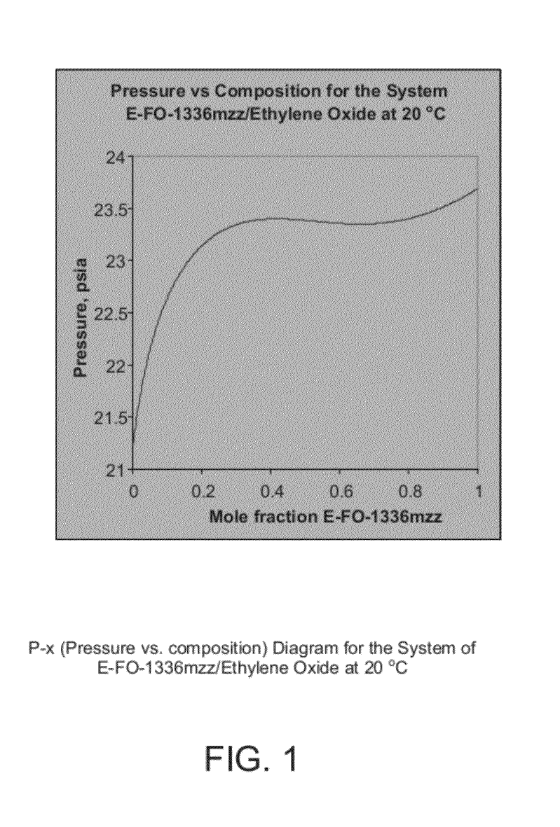

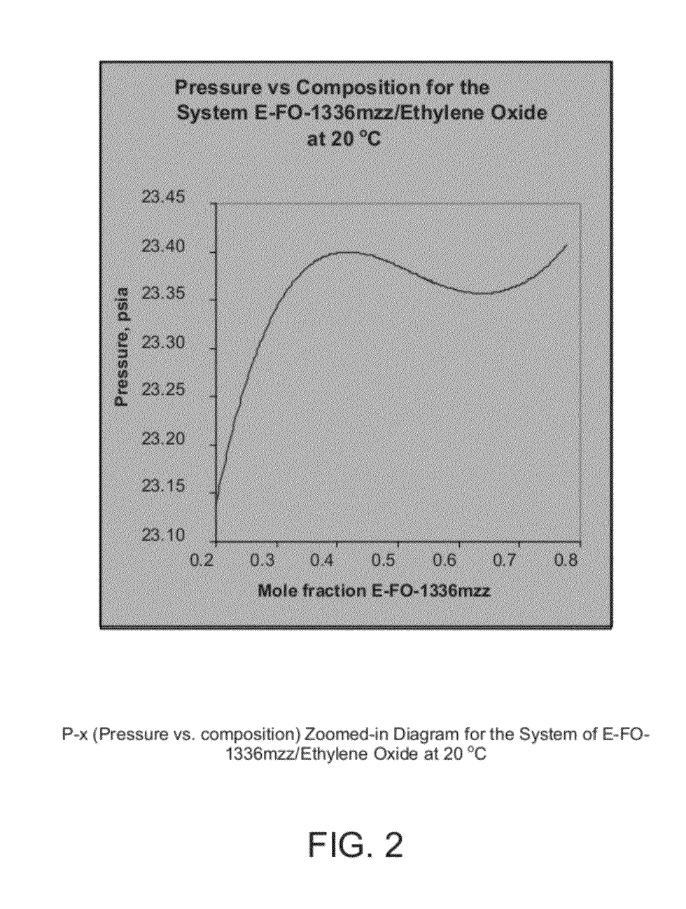

The vapor pressure measured versus the compositions in the PTx cell for E-FO-1336mzz/ethylene oxide mixture is shown in FIG. 1 and FIG. 2, which graphically illustrate the formation of double azeotropic and azeotrope-like compositions at about 20° C. consisting essentially of E-FO-1336mzz and ethylene oxide as indicated by a mixture of about 41.6 mole % E-1,1,1,4,4,4-hexafluoro-2-butene and 58.4 mole % ethylene oxide having a maximum pressure of about 23.40 psia (161.3 kPa), and a mixture of about 65.6 mole % E-1,1,1,4,4,4-hexafluoro-2-butene and 34.4 mole % ethylene oxide having a minimum pressure of about 23.35 psia (160.9 kPa).

Based upon these findings, it has been calculated that E-FO-1336mzz and ethylene oxide form azeotropic or azeotrope-like compositions at temperatures of from about −40° C. to about 120° C. It has also been calculated that E-FO-1336mzz and ethylene oxide form double azeotropic or double azeotrope-like compositions at temperatures of from about 0° C. to about 120° C.

It was found that E-FO-1336mzz and ethylene oxide form azeotropic compositions ranging from about 9.2 mole percent to about 79.6 mole percent E-FO-1336mzz and from about 90.8 mole percent to about 20.4 mole percent ethylene oxide.

It was found that E-FO-1336mzz and ethylene oxide form azeotropic compositions at a temperature of from about −40° C. to about 120° C.

It was found that E-FO-1336mzz and ethylene oxide form azeotropic compositions at a pressure of from about 1.41 psia (9.72 kPa) to about 303.4 psia (2091 kPa).

It was also found that E-FO-1336mzz and ethylene oxide form double azeotropic compositions ranging from about 9.2 mole percent to about 79.6 mole percent E-FO-1336mzz and from about 90.8 mole percent to about 20.4 mole percent ethylene oxide.

It was also found that E-FO-1336mzz and ethylene oxide form double azeotropic compositions at a temperature of from about 0° C. to about 120° C.

It was also found that E-FO-1336mzz and ethylene oxide form double azeotropic compositions at a pressure of from about 10.7 psia (73.8 kPa) to about 303.4 psia (2091 kPa).

Some embodiments of azeotropic and double azeotropic compositions are listed in Table 1.

TABLE 1
Azeotropic Compositions

| Azeotropic | Azeotropic | | Ethylene |
|---|---|---|---|
| Temperature | Pressure | E-FO-1336mzz | Oxide |
| (° C.) | (psia) | (mole %) | (mole %) |
| −40 | 1.41 ^{a} | 0.378 | 0.622 |
| −20 | 4.29 ^{a} | 0.396 | 0.604 |
| 0 | 10.78 ^{a} | 0.408 | 0.592 |
| 0 | 10.70 ^{b} | 0.796 | 0.204 |
| 20 | 23.40 ^{a} | 0.416 | 0.584 |
| 20 | 23.35 ^{b} | 0.660 | 0.340 |
| 40 | 45.28 ^{a} | 0.414 | 0.586 |
| 40 | 45.30 ^{b} | 0.556 | 0.444 |
| 60 | 80.06 ^{a} | 0.376 | 0.624 |
| 60 | 80.03 ^{b} | 0.498 | 0.502 |
| 80 | 131.62 ^{a} | 0.291 | 0.709 |
| 80 | 131.40 ^{b} | 0.487 | 0.513 |
| 100 | 204.3 ^{a} | 0.193 | 0.807 |
| 100 | 203.0 ^{b} | 0.493 | 0.507 |
| 120 | 303.4 ^{a} | 0.092 | 0.908 |
| 120 | 297.1 ^{b} | 0.512 | 0.488 |

^{a} the azeotropic composition forms at a maximum pressure.
^{b} the azeotropic composition forms at a minimum pressure.

Additionally, azeotrope-like compositions containing E-FO-1336mzz and ethylene oxide may also be formed. Such azeotrope-like compositions exist around azeotropic compositions. Some embodiments of azeotrope-like compositions are listed in Table 2. Additional embodiments of azeotrope-like compositions are listed in Table 3.

TABLE 2
Azeotrope-like compositions

| | | Mole Percent |
|---|---|---|
| COMPONENTS | T (° C.) | Range |
| E-FO-1336mzz/Ethylene oxide | −40 | 1-99/99-1 |
| E-FO-1336mzz/Ethylene oxide | −20 | 1-99/99-1 |
| E-FO-1336mzz/Ethylene oxide | 0 | 1-99/99-1 |
| E-FO-1336mzz/Ethylene oxide | 20 | 1-99/99-1 |
| E-FO-1336mzz/Ethylene oxide | 40 | 1-99/99-1 |
| E-FO-1336mzz/Ethylene oxide | 60 | 1-99/99-1 |
| E-FO-1336mzz/Ethylene oxide | 80 | 1-99/99-1 |
| E-FO-1336mzz/Ethylene oxide | 100 | 1-99/99-1 |
| E-FO-1336mzz/Ethylene oxide | 120 | 1-99/99-1 |
| E-FO-1336mzz/Ethylene oxide | 140 | 1-99/99-1 |

TABLE 3
Azeotrope-like compositions

| | | Mole Percent |
|---|---|---|
| COMPONENTS | T (° C.) | Range |
| E-FO-1336mzz/Ethylene oxide | −40 | 5-95/95-5 |
| E-FO-1336mzz/Ethylene oxide | −20 | 5-95/95-5 |
| E-FO-1336mzz/Ethylene oxide | 0 | 5-95/95-5 |
| E-FO-1336mzz/Ethylene oxide | 20 | 5-95/95-5 |
| E-FO-1336mzz/Ethylene oxide | 40 | 5-95/95-5 |
| E-FO-1336mzz/Ethylene oxide | 60 | 5-95/95-5 |
| E-FO-1336mzz/Ethylene oxide | 80 | 5-95/95-5 |
| E-FO-1336mzz/Ethylene oxide | 100 | 5-95/95-5 |
| E-FO-1336mzz/Ethylene oxide | 120 | 5-95/95-5 |
| E-FO-1336mzz/Ethylene oxide | 140 | 5-95/95-5 |

The azeotropic or azeotrope-like compositions of the present invention can be prepared by any convenient method including mixing or combining the desired amounts. In one embodiment of this invention, an azeotropic or azeotrope-like composition can be prepared by weighing the desired component amounts and thereafter combining them in an appropriate container. In some embodiments, the components of the azeotropic or azeotrope-like composition are physically combined as liquefied gases in cylinders.

The azeotropic or azeotrope-like compositions of the present invention can be used in a wide range of applications, including their use as aerosol propellants, refrigerants, solvents, cleaning agents, sterilant mixtures, blowing agents (foam expansion agents) for thermoplastic and thermoset foams, heat transfer media, gaseous dielectrics, fire extinguishing and suppression agents, power cycle working fluids, polymerization media, particulate removal fluids, carrier fluids, buffing abrasive agents, and displacement drying agents.

One embodiment of this invention provides a process for preparing a thermoplastic or thermoset foam. The process comprises using an azeotropic or azeotrope-like composition as a blowing agent, wherein said azeotropic or azeotrope-like composition consists essentially of (a) E-FO-1336mzz and (b) ethylene oxide; wherein the ethylene oxide is present in an effective amount to form an azeotropic or azeotrope-like mixture with E-FO-1336mzz.

Another embodiment of this invention provides a process for producing refrigeration. The process comprises condensing an azeotropic or azeotrope-like composition and thereafter evaporating said azeotropic or azeotrope-like composition in the vicinity of the body to be cooled, wherein said azeotropic or azeotrope-like composition consists essentially of (a) E-FO-1336mzz and (b) ethylene oxide; wherein the ethylene oxide is present in an effective amount to form an azeotropic or azeotrope-like mixture with E-FO-1336mzz.

Another embodiment of this invention provides a process using an azeotropic or azeotrope-like composition as a solvent, wherein said azeotropic or azeotrope-like composition consists essentially of (a) E-FO-1336mzz and (b) ethylene oxide; wherein the ethylene oxide is present in an effective amount to form an azeotropic or azeotrope-like mixture with E-FO-1336mzz.

Another embodiment of this invention provides a process for producing an aerosol product. The process comprises using an azeotropic or azeotrope-like composition as a propellant, wherein said azeotropic or azeotrope-like composition consists essentially of (a) E-FO-1336mzz and (b) ethylene oxide; wherein the ethylene oxide is present in an effective amount to form an azeotropic or azeotrope-like mixture with E-FO-1336mzz.

Another embodiment of this invention provides a process using an azeotropic or azeotrope-like composition as a heat transfer media, wherein said azeotropic or azeotrope-like composition consists essentially of (a) E-FO-1336mzz and (b) ethylene oxide; wherein the ethylene oxide is present in an effective amount to form an azeotropic or azeotrope-like mixture with E-FO-1336mzz.

Another embodiment of this invention provides a process for extinguishing or suppressing a fire. The process comprises using an azeotropic or azeotrope-like composition as a fire extinguishing or suppression agent, wherein said azeotropic or azeotrope-like composition consists essentially of (a) E-FO-1336mzz and (b) ethylene oxide; wherein the ethylene oxide is present in an effective amount to form an azeotropic or azeotrope-like mixture with E-FO-1336mzz.

Another embodiment of this invention provides a process using an azeotropic or azeotrope-like composition as dielectrics, wherein said azeotropic or azeotrope-like composition consists essentially of (a) E-FO-1336mzz and (b) ethylene oxide; wherein the ethylene oxide is present in an effective amount to form an azeotropic or azeotrope-like mixture with E-FO-1336mzz.

Another embodiment of this invention provides a method for sterilizing an article. Azeotropic or azeotrope-like compositions comprising E-FO-1336mzz and ethylene oxide may be particularly useful in methods for sterilizing, due to the sterilant composition maintaining a constant concentration of E-FO-1336mzz and ethylene oxide, which will remain non-flammable for the full duration of use.

In some embodiments, the sterilant composition consisting essentially of E-FO-1336mzz and ethylene oxide may further comprise inert propellants, which may be used to increase the pressure in a sterilant container in order to propel the sterilant composition into the sterilization chamber. Suitable propellants include nitrogen, carbon dioxide, argon and trifluoromethane and the like.

In one embodiment, is provided a method for sterilizing an article comprising contacting the article to be sterilized with an effective amount of an azeotropic or azeotrope-like composition consisting essentially of E-FO-1336mzz and ethylene oxide as described herein. In another embodiment, is provided a method for sterilizing an article comprising contacting the article to be sterilized with an azeotropic or azeotrope-like composition consisting essentially of E-FO-1336mzz and ethylene oxide composition as described herein under conditions and for a period of time as to be effective in achieving the desired degree of sterility. In another embodiment, the method comprises placing the articles to be sterilized in a vessel, evacuating the air from the vessel, humidifying the vessel, and contacting the articles with the an azeotropic or azeotrope-like composition consisting essentially of E-FO-1336mzz and ethylene oxide composition for an effective period of time.

The azeotropic or azeotrope-like composition consisting essentially of E-FO-1336mzz and ethylene oxide may be used to sterilize a great many articles, including but not limited to medical equipment and materials, including diagnostic endoscopes, plastic goods such as syringes, gloves, test tubes, incubators and pacemakers; rubber goods such as tubing, catheters and sheeting; instruments such as needles, scalpels and oxygen tests; and other items such as dilators, pumps, motors and intra ocular lenses. In another embodiment, the azeotropic or azeotrope-like composition consisting essentially of E-FO-1336mzz and ethylene oxide may be used as a fumigant for items outside the medical field including but not limited to certain food stuffs, such as spices, and other items such as furs, bedding, paper goods, and transportation equipment such as the cargo area of airplanes, trains, and ships. Ethylene oxide penetrates well, moving through paper, cloth, and some plastic films and can therefore be used to sterilize articles sealed inside certain containers including polyethylene bags.

The azeotropic or azeotrope-like composition consisting essentially of E-FO-1336mzz and ethylene oxide may be effective against all forms of life, particularly unwanted insects, bacteria, virus, molds, fungi, and other microorganisms. An azeotropic or azeotrope-like composition consisting essentially of E-FO-1336mzz and ethylene oxide may be used to process sensitive instruments which cannot be adequately sterilized by other methods. Ethylene oxide containing compositions are known to kill all known viruses, bacteria and fungi, including bacterial spores and is satisfactory for most medical materials, even with repeated use.

In some embodiments, when in use, the liquid phase of an azeotropic or azeotrope-like composition consisting essentially of E-FO-1336mzz and ethylene oxide can be expelled into a heat exchanger where it is vaporized and then introduced into a sterilizing chamber. In other embodiments, the cylinder can be heated directly to generate vapor.

In some embodiments, the sterilization method may be carried out in a rigid chamber or box. In other embodiments, the sterilization method may be carried out in a flexible chamber or bag housed within a chamber or box.

In some embodiments, the method for sterilization is carried out between about 25° C. to about 70° C. In other embodiments, the sterilization is carried out between about 30° C. and about 60° C. In yet other embodiments, the sterilization is carried out between about 54° C. and 60° C.

A moist microorganism is more susceptible to the reaction of the sterilant composition, therefore in some embodiments, water vapor is used to provide a relative humidity above about 20 percent. In some embodiments, the relative humidity used for sterilization is above about 30 percent. In some embodiments, the relative humidity used for sterilization is from about 30 percent to about 80 percent.

In some embodiments, an effect period of time for sterilizing will depend upon a number of factors including temperature, pressure, relative humidity, the specific sterilant composition employed and the material being sterilized. In other embodiments, sterilization of some porous articles may require shorter contact times than do articles sealed in polyethylene bags. Further, in another embodiment, certain bacteria are especially resistant and may thus require longer contact times for sterilization.

In some embodiments, the time required for sterilization may range from about 2 hours to 24 hours. In other embodiments the time required for sterilization may range from about 3 hours to 12 hours. In other embodiments, the time required for sterilization may be at least 3 hours.

1. An azeotropic composition consisting essentially of:
(a) E-1,1,1,4,4,4-hexafluoro-2-butene; and
(b) ethylene oxide; wherein the ethylene oxide is present in an effective amount to form an azeotropic combination with the E-1,1,1,4,4,4-hexafluoro-2-butene.

2. The azeotropic composition of claim 1 wherein said azeotropic composition consists essentially of about 9.2 to about 79.6 mole percent E-1,1,1,4,4,4-hexafluoro-2-butene and about 90.8 to about 20.4 mole percent ethylene oxide.

3. The azeotropic composition of claim 1 wherein said azeotropic composition forms at a temperature of from about −40° C. to about 120° C.

4. The azeotropic composition of claim 1 wherein said azeotropic composition forms at a pressure of from about 1.41 psia (9.72 kPa) to about 303.4 psia (2091 kPa).

5. An azeotrope-like composition consisting essentially of:
(a) E-1,1,1,4,4,4-hexafluoro-2-butene; and
(b) ethylene oxide; wherein the ethylene oxide is present in an effective amount to form an azeotrope-like combination with the E-1,1,1,4,4,4-hexafluoro-2-butene.

6. The azeotrope-like composition of claim 5 wherein said azeotrope-like composition consists essentially of about 5 to about 95 mole percent E-1,1,1,4,4,4-hexafluoro-2-butene and about 95 to about 5 mole percent ethylene oxide at a temperature of from about −40° C. to about 140° C.

7. The azeotrope-like composition of claim 5 wherein said azeotrope-like composition consists essentially of about 1 to about 99 mole percent E-1,1,1,4,4,4-hexafluoro-2-butene and about 99 to about 1 mole percent ethylene oxide at a temperature of from about −40° C. to about 140° C.

8. A double azeotropic composition consisting essentially of:
(a) E-1,1,1,4,4,4-hexafluoro-2-butene; and
(b) ethylene oxide; wherein the ethylene oxide is present in an effective amount to form a double azeotropic combination with the E-1,1,1,4,4,4-hexafluoro-2-butene.

9. The double azeotropic composition of claim 8 wherein said double azeotropic composition consists essentially of about 9.2 to about 79.6 mole percent E-1,1,1,4,4,4-hexafluoro-2-butene and about 90.8 to about 20.4 mole percent ethylene oxide.

10. The double azeotropic composition of claim 8 wherein said double azeotropic composition forms at a temperature of from about 0° C. to about 120° C.

11. The double azeotropic composition of claim 8 wherein said double azeotropic composition forms at a pressure of from about 10.7 psia (73.8 kPa) to about 303.4 psia (2091 kPa).

12. A process for preparing a thermoplastic or thermoset foam comprising using an azeotropic or azeotrope-like composition as a blowing agent, wherein said azeotropic or azeotrope-like composition consists essentially of:
(a) E-1,1,1,4,4,4-hexafluoro-2-butene; and
(b) ethylene oxide; wherein the ethylene oxide is present in an effective amount to form an azeotropic or azeotrope-like combination with the E-1,1,1,4,4,4-hexafluoro-2-butene.

13. A process for producing refrigeration comprising condensing an azeotropic or azeotrope-like composition and thereafter evaporating said azeotropic or azeotrope-like composition in the vicinity of the body to be cooled, wherein said azeotropic or azeotrope-like composition consists essentially of:
(a) E-1,1,1,4,4,4-hexafluoro-2-butene; and
(b) ethylene oxide; wherein the ethylene oxide is present in an effective amount to form an azeotropic or azeotrope-like combination with the E-1,1,1,4,4,4-hexafluoro-2-butene.

14. A process comprising using an azeotropic or azeotrope-like composition as a solvent, wherein said azeotropic or azeotrope-like composition consists essentially of:
(a) E-1,1,1,4,4,4-hexafluoro-2-butene; and
(b) ethylene oxide; wherein the ethylene oxide is present in an effective amount to form an azeotropic or azeotrope-like combination with the E-1,1,1,4,4,4-hexafluoro-2-butene.

15-20. (canceled)
